(19) United States
(12) Patent Application Publication
(10) Pub. No.: US 2009/0176891 A1
(43) Pub. Date: Jul. 9, 2009

(54) DENTAL COMPOSITION AND METHOD OF USE

(76) Inventors: SAMI CHOGLE, Cleveland Heights, OH (US); Sohel Shaikh, Sugarland, TX (US); Andre Mickel, Cleveland Heights, OH (US); Syed Qutubuddin, Pepper Pike, OH (US); Mohan Sankaran, University Hieghts, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/330,144

(22) Filed: Dec. 8, 2008

Related U.S. Application Data
(60) Provisional application No. 60/992,875, filed on Dec. 6, 2007.

(51) Int. Cl. A61K 6/083; C08J 3/28; A01P 1/00; A61C 5/00

(52) U.S. Cl. 514/772.6; 522/112; 522/77; 522/71; 433/217.1

(57) ABSTRACT

A dental composition for sealing a portion of a tooth includes a liquid acrylic or acrylate monomer, an acrylic or acrylate polymer that is soluble in the liquid acrylic or acrylate monomer, a photo-initiator for cross-linking the liquid acrylic or acrylate monomer, and a nanoparticle material dispersed within the composition.

### RELATED APPLICATION

This application claims priority from U.S. Provisional Application No. 60/992,875, filed Dec. 6, 2007, the subject matter, which is incorporated herein by reference.

### TECHNICAL FIELD

The present invention relates generally to a nanocomposite composition, and more particularly to a nanocomposite composition for endodontic and bone repair applications.

### BACKGROUND OF THE INVENTION

In the field of endodontics, success in root canal therapy is based on elimination or breakdown of the microbial ecology in the root canal system through biomechanical cleaning and shaping, as well as hermetic three-dimensional obturation. As part of the biomechanical preparation of the tooth, the root canal is typically cleansed or “filed” and chemically rinsed with an anti-microbial agent. With complex anatomy like that of the root canal system, this preparation sometimes fails to break the microbial ecosystem. This can lead to a chronic infection and predispose a tooth to periapical breakdown, eventual loss of periodontal attachment, and ultimately tooth loss.

As a possible treatment, root-end surgery (apicoectomy) is performed when there is an incidence, persistence, or enlargement of root-end pathology following endodontic treatment. Root-end surgery may be advocated when a marked overextension of root canal filling material penetrates into the surrounding bone and interferes with healing. Additionally, root-end surgery may be advocated when the apical portion of the root canal system with periapical pathosis cannot be adequately cleaned, shaped and obturated. The procedure commonly involves surgical exposure and resection of the apical end of the infected root. The placement of a root-end filling (retrofill) material is an additional procedure following root-end resection.

The primary function of a root-end filling is to improve the seal of the root canal system. It is important that the retrograde filling material can prevent leakage of infected substances from the root canal to the periapical tissue. Insufficient retrograde sealing of root canals after apicoectomy is considered a major cause of surgical endodontic failures. Retrofilling materials have also been used to seal resorptive defects, and repair root perforations, demonstrating their versatility and vast importance in the specialty of endodontics.

For over a century, amalgam has been the most commonly used material for root-end fillings. Newer root-end filling materials, such as SuperEBA, GERISTORE, or MTA are also used for root-end fillings. The use of such materials has many disadvantages including initial marginal leakage, corrosion, mercury contamination of peripheral tissues, moisture sensitivity to some alloys, the need for a retentive undercut preparation, and staining of hard and soft tissues. Additionally, the use of such materials can cause severe tissue reactions, possibly due to substantial leakage, which can in turn lead to low success rates in endodontic surgery.

### SUMMARY OF THE INVENTION

The present invention relates generally to dental compositions and methods for tooth repair, and more particularly to a nanocomposite composition for sealing a portion of a tooth. According to one aspect of the present invention, a dental composition for sealing a portion of a tooth can include a liquid acrylic or acrylate monomer, an acrylic or acrylate polymer that is soluble in the liquid acrylic or acrylate monomer, a photo-initiator for cross-linking the liquid acrylic or acrylate monomer, and a nanoparticle material dispersed within the composition.

According to another aspect of the present invention, the dental composition for sealing a portion of a tooth can include, by weight of the dental composition, about 20% to about 60% of a liquid acrylic or acrylate monomer, about 50% to about 60% of an acrylic or acrylate polymer that is at least partially soluble in the liquid acrylic or acrylate monomer, about 0.05% to about 10% of a photo-initiator for cross-linking the liquid acrylic or acrylate monomer, and about 0% to about 5% of a nanoparticle material dispersed within the composition.

Another aspect of the present invention relates to a method for sealing a portion of a tooth. The method can include administering to the portion of the tooth at least one application of a dental composition. The dental composition can include a liquid acrylic or acrylate monomer, an acrylic or acrylate polymer that is soluble in the liquid acrylic or acrylate monomer, a photo-initiator, and a nanoparticle material. The dental composition can then be photo-cured so that the liquid acrylic or acrylate monomer and the soluble acrylic or acrylate polymer are cross-linked and form a three-dimensional polymer matrix having the nanoparticle material dispersed therein.

### BRIEF DESCRIPTION OF THE DRAWINGS

The foregoing and other features of the present invention will become apparent to those skilled in the art to which the present invention relates upon reading the following description with reference to the accompanying drawings.

FIG. 1 is a cross-sectional view of a tooth showing a root canal and an infection at an apex of the tooth.

FIG. 2 is a cross-sectional view of the tooth in FIG. 1 showing a dental tool removing the infected tissue and a portion of the apex.

FIG. 3 is a cross-sectional view of the tooth in FIG. 2 showing a dental composition being administered to the root canal at the removed portion of the apex.

FIG. 4 is a cross-sectional view of the tooth in FIG. 3 showing the dental composition being cured by a radiant light source.

FIG. 5 is a graph comparing the sealability of different polymer nanocomposites, a control, and a commercially available compomer.

FIG. 6 is graph showing the antimicrobial efficacy of various dose strengths of CHX on bacterial growth on agar plates.

FIG. 7 illustrates a SEM micrograph showing MTA with no dentin tubular penetration.

FIG. 8 illustrates a SEM micrograph showing GERISTORE with some dentin tubular penetration.

FIG. 9 illustrates a SEM micrograph showing dentin tubular penetration of polymer nano composite with 1% organoclay content (Inset: higher magnification).

FIG. 10 illustrates a SEM micrograph showing dentin tubular penetration of polymer nano composite with 2% organoclay content.

FIG. 11 illustrates a chart showing shear stress for PNC samples compared to MTA and GERISTORE in the presence of dentinal smear lay.

FIG. 12 illustrates a chart showing shear stress for PNC samples compared to MTA and GERISTORE in the absence of dentinal smear layer.

FIG. 13 illustrates plots of XRD spectra of (A) Na-MMT and C18-MMT (OC) and (B) PNC with clay loading of 0.5 to 2.5%.

FIG. 14 illustrates TEM Micrographs of PNCs at varying OC content (50 nm scale) (A) 0.5% OC (B) 1.0% OC(C) 1.5% OC (D) 2.0% OC.

FIG. 15 illustrates ¹H NMR spectra comparison of MM1+I2 (top spectrum) and MM1+I4 (bottom spectrum).

### DETAILED DESCRIPTION

The present invention relates generally to dental compositions and methods for tooth repair, and more particularly to a nanocomposite composition for sealing a portion of a tooth. The dental composition of the present invention is photo-curable, exhibits minimal shrinkage upon curing, is biologically inert, and mitigates initial and long-term leakage of root-end filling material from a portion of a tooth (e.g., a root canal). Moreover, the dental composition can provide controlled release of therapeutic agents from the dental composition over an extended time period.

The dental composition of the present invention provides a polymerizable composition for use in a variety of endodontic procedures. Although the use of the dental composition is described below in the context of apical plug formation during an apicoectomy or root canal surgery, it will be appreciated that the dental composition may also be used in any other type of surgical or non-surgical endodontic procedure.

The dental composition of the present invention is effective for not only sealing the root canal from the ingress of fluids and bacteria from the crown and/or periodontal tissue, but also for preventing or mitigating initial and long-term leakage of root-end filling material from a portion of a tooth. As described in more detail below, the dental composition (or a component thereof) can penetrate the microporous structure of the tooth (i.e., the dental tubules) such that a tight seal is formed. This helps to ensure the mechanical integrity of the treated tooth and protect the surrounding dental tissue from being infected by bacteria. Additionally, since the dental composition is photo-curable, a dental care provider (e.g., a dentist or endodontist) may have greater control over the time it takes to provide sufficient curing of the dental composition and thereby permit more precise placement of the dental composition and/or a prosthetic covering.

The dental composition of the present invention can include a liquid acrylic or acrylate monomer, an acrylic or acrylate polymer that is soluble in the liquid acrylic or acrylate monomer, a photo-initiator for cross-linking the liquid acrylic or acrylate monomer, and a nanoparticle material dispersed within the composition. As used herein, the term “acrylic” refers to any molecule or compound that contains an acryl group derived from acrylic acid. Additionally, the term “acrylate” as used herein refers to an ion of acrylic acid having the formula CH₂═CHCOO⁻.

The liquid acrylic or acrylate monomer can include any acrylic or acrylate monomer that can be used in dental applications, including endodontic applications. Examples of the liquid acrylic or acrylate monomer can include urethane dimethacrylate (UDMA), p-hydroxyphenyl methacrylamide, butanediol dimethacrylate, 2,2-bis[p-(2-hydroxy-3-methacryloxypropoxy)phenyl]propane (bisGMA), triethylene glycol dimethacrylate (TEGDMA), and hydroxyl ethyl methacrylate (HEMA). The liquid acrylic or acrylate monomer may be included in the dental composition in the amount of up to about 95% by weight of the composition, for example in a range from about 5% to about 80% by weight, or in a range from about 20% to about 60% by weight of the composition.

The soluble acrylic or acrylate polymer can include any acrylic or acrylate polymer that is soluble in the liquid acrylic or acrylate monomer and that is effective to mitigate adverse shrinkage of the dental composition upon curing. It will be appreciated that the soluble acrylic or acrylate polymer can be partially or completely soluble in the liquid acrylic or acrylate monomer. As described in further detail below, the concentration of the liquid acrylic or acrylate monomer in the dental composition can be less than the concentration of the soluble acrylic or acrylate polymer. In this case, a homogenous or “workable” paste may be formed. The soluble acrylic or acrylate polymer in accordance with the present invention can be at least partially hydrophilic and/or include constituents, which make an otherwise hydrophobic dental composition more retentive within dental tissue.

Examples of the soluble acrylic or acrylate polymer can include, but are not limited to, methacrylates, alkylhydroxy methacrylates, alkylamino methacrylates, and derivatives thereof. More specific examples of the soluble acrylic or acrylate polymer can include glycidyl dimethacrylate, 2-hydroxy ethyl methacrylate, 3-hydroxy propyl methacrylate, 4-hydroxy butyl methacrylate, triethylene glycol dimethacrylate, polyethylene glycol dimethacrylate, and poly(methyl methcrylate) (PMMA). The soluble acrylic or acrylate polymer can be included in the dental composition at a concentration effective to mitigate adverse shrinkage of the dental composition upon curing. This can include a concentration ranging from about 10% to about 250% by weight of the composition, for example from about 30% to about 150% by weight, or from about 50% to about 60% by weight of the composition.

The photo-initiator can include any photo-initiator that can initiate or induce polymerization of the liquid acrylic or acrylate monomer. Examples of the photo-initiator can include camphorquinone, benzoin methyl ether, 2-hydroxy-2-methyl-1-phenyl-1-propanone, diphenyl 2,4,6-trimethylbenzoyl phosphine oxide, benzoin ethyl ether, benzophenone, 9,10-anthraquinone, N,N-dimethylaminoethyl methacrylate, ethyl-4-N,N-dimethylaminobenzoate, diphenyliodonium chloride and derivatives thereof. The photo-initiator can be included in the dental composition in an amount of about 0.05% to about 10% by weight of the composition, and for example in an amount of about 1% to about 2% by weight of the composition. The photo-initiator can also be a combination of agents such as camphoquinone and an amine, for example. A high degree of photopolymerization is desirable to reduce any residual liquid acrylic or acrylate monomer (e.g., HEMA) content in the dental composition. A low residual monomer content may be obtained by using a combination of two or more different photo-initiators.

The nanoparticle material can include any nanoparticle material that is dispersed in the dental composition and enhances the material properties (e.g., the stiffness, strength, dimensional stability, and thermal properties) of the cured dental composition. The nanoparticle material can have at least one ultrafine dimension of less than about 100 nanometers.

In one aspect of the invention, the nanoparticle material can be provided in the dental composition at an amount effective to a uniform dispersion of the nanoparticles and enhance the material properties of the cured dental composition. By way of example, the nanoparticle material can be dispersed within the dental composition in an amount ranging from about 0% to about 5% by weight, and for example in an amount of about 2% by weight of the dental composition.

In an example of the present invention, the nanoparticle material can include an organically-modified hydrophilic clay (organoclay). The organoclay can include, but is not limited to, organically-modified montmorillonite, fluorohectorite, bentonite, fluoromica, and layered double hydroxides. The nanoscale structure and large interfacial area between a given polymer and the organoclay make polymer-organoclay compositions effective for endodontic procedures because such compositions exhibit enhanced mechanical and thermal properties, reduced gas permeability, and improved drug elution characteristics. Additionally, polymer-organoclay compositions may be particularly attractive for potential applications where enhanced barrier properties as well as physical properties are desired.

In another example of the present invention, the nanoparticle material can include a carbon nanotube (CNT). Generally, CNTs have unique mechanical properties, such as high stiffness and axial strength, as a result of their cylindrical graphite structure. CNTs also possess exceptionally high Young's moduli in the terapascal range, which are much higher than those typically found in stainless steel and carbon fibers. Including CNTs having smaller diameters and larger aspect ratios can yield high strength cured dental compositions. CNTs may be dispersed in polymer matrices to obtain substantial improvements in matrix properties such as tensile strength, modulus, and dimensional stability (see, e.g., Bower, C. et al.; Applied Physics Letters 1999, 74:3317-3319; Velasco-Santos, C. et al.; Chem. Mater. 2003, 15:4470-4475; Li, S. et al.; Chem. Mater. 2005, 17:130-135; Liu, C. et al., Applied Physics Letters 2005, 86:1231061-3; Guo, H. et al., Polymer 2005, 46:3001-3005; and Wang, S. et al., Advanced Functional Materials, 2007, 17(1):87-92). For example, significant attention has been given to achieving a homogeneous dispersion of CNTs in a polymer matrix.

In another aspect of the present invention, the dental composition can include by weight of the dental composition about 20% to about 60% of a mixture of acrylate or acrylic monomers, about 40% to about 80% of an acrylate or acrylic polymer that is soluble in the mixture of acrylate or acrylic monomers; about 0.05% to about 10% of a photo or chemical initiator for cross-linking the liquid acrylic or acrylate monomers; and about 0.1% to about 5% of a nanoparticle material dispersed within the composition.

In another aspect of the present invention, the dental composition can include by weight of the dental composition about 20% to about 60% of a mixture of bisGMA, TEGDMA, and HEMA; about 40% to about 80% of a PMMA that is soluble in the mixture of bisGMA, TEGDMA, and HEMA; about 0.05% to about 10% of a mixture of camphorquinone and N,N-dimethylaminobenzoate for cross-linking the liquid acrylic or acrylate monomers; and about 0.1% to about 5% of an organoclay or carbon nanotube dispersed within the composition.

It will be appreciated that the dental composition can include other components in addition to those described above. For example, the dental composition can include one or more anti-microbial agents to assist in cleansing and sterilizing the root canal to prevent later infection. Examples of anti-microbial agents can include organohalogens, antibiotics, alkali metal hydroxides, alkaline earth metal oxides, and alkaline earth metal hydroxides. Examples of antibacterial organohalogens can include 1,1′-hexamethylene bis(5(p-chlorophenyl)biguanide), cetyl pyridinium chloride, benzalkonium chloride, and cetyl pyridinium bromide. Examples of antibiotics can include 4′ sulfamoylsulfanilanilide, 3-amino-6-(2-(5-nitro-2-furyl)vinyl)pyridazine, trans-pseudomonic acid, xanthomycin, alpha-amino-p-toluene sulfonamide, alpha-azido benzyl penicillin, penicillin O, penicillin N, monopropionyl erthromycin, erythromycin 9(O-((2-methoxy ethoxy)methyl)oxime, and members of the tetracycline family (e.g., doxycycline, minocycline, etc.). Examples of alkali metal hydroxides can include sodium hydroxide and lithium hydroxide. Examples of alkaline earth metal oxides can include calcium oxide, magnesium oxide, barium oxide, and strontium oxide. Examples of alkaline earth metal hydroxides can include calcium hydroxide, magnesium hydroxide, barium hydroxide, and strontium hydroxide.

The anti-microbial agent may be included in the dental composition in an amount from about 0.001% to about 30% by weight of the composition, preferably in a range from about 1% to about 10% by weight, and most preferably in an amount of about 2% by weight of the composition.

It should also be appreciated that the dental composition can additionally or alternatively include a radio-opaque filler. Radio-opaque fillers can be included in the dental composition to determine how well the dental composition has filled a root canal, for example. Examples of fillers that can provide increased radio-opacity can include bismuth salts such as bismuth chloride, silver and silver salts such as silver chloride, barium salts such as barium sulfate or barium chloride, tungsten salts, titanium dioxide, and strontium salts such as strontium sulfate and strontium chloride. These and other fillers, such as silicon dioxide and calcium phosphate tribasic, may also be used to minimize polymerization shrinkage and/or the total heat potential associated with polymerization of the dental composition.

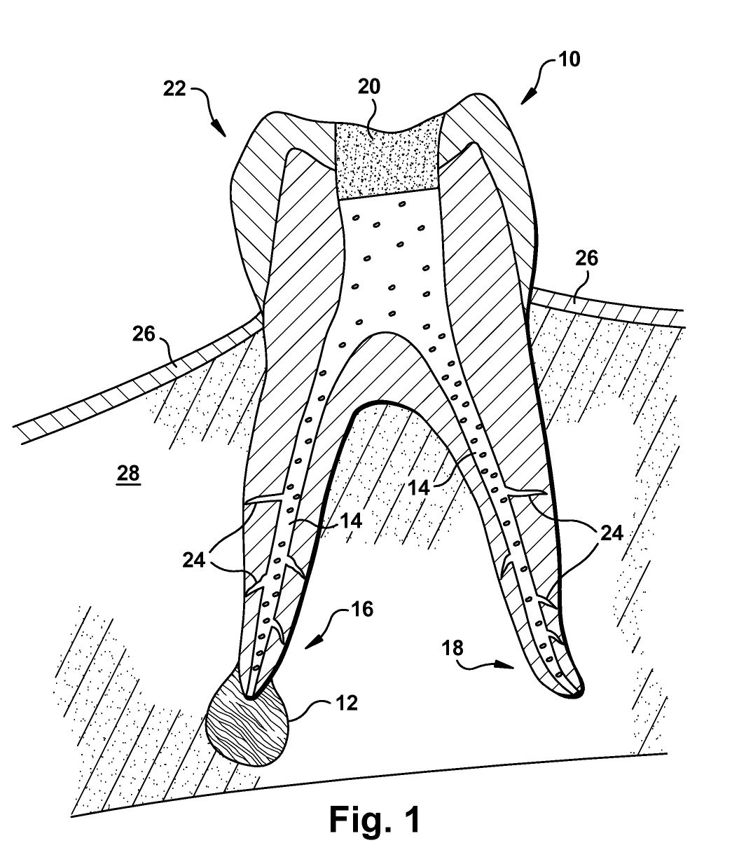

Another aspect of the invention relates to a method for sealing a portion of a tooth 10 (FIGS. 1-4). FIG. 1 depicts a tooth 10 that has been subjected to a root canal procedure and has developed an infection 12 as a result of a bacterial infection after the procedure. The tooth 10 includes a pair of root canals 14, each of which includes root filler material (dotted) and terminates at a first apex 16 and a second apex 18 that extend through the bottom of the root. A seal 20 is located at the crown 22 of the tooth 10. Lateral canals 24 extend from the root canals and provide communication between the root canals. In addition, the gum tissue 26 and bone tissue 28 surrounding the tooth 10 are shown.

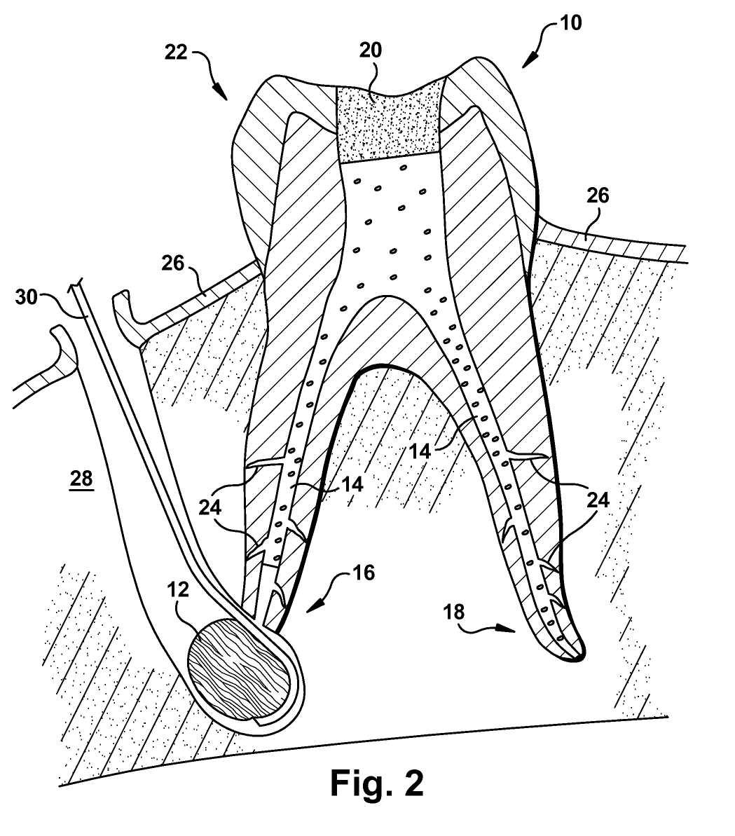

To fill the portion of the tooth 10, an apicoectomy can be performed using a microscopic surgical approach as known in the art. For example, one step of the method can include preparing the tooth 10 for surgery by first incising and lifting away the gum tissue 26 from the tooth so that the first apex 16 will be accessible. Next, accesses to the first apex 16 and the abscess 12 can be obtained using a drill (not shown) or other acceptable device. Once access to the first apex 16 is obtained, an appropriate dental tool 30 can be used to remove the infected tissue 12 and a portion of the first apex (FIG. 2). As shown in FIG. 2, the dental tool 30 can be used to remove not only the entire infected tissue 12, but also a few millimeters of the first apex 16 as well as some of the surrounding periodontal tissue.

After preparing the tooth 10 as shown in FIG. 2, the dental composition can be prepared as described in Example 2 and as shown in Table 2a below. For example, the dental composition can comprise about 45% by weight of bisGMA, about 45% by weight of TEGDMA, about 10% by weight of HEMA, about 0.2% by weight of camphorquinone, about 0.8% by weight of ethyl-4-N,N-dimethylaminobenzoate, about 150% by weight of PMMA, and about 1% by weight of organically-modified montmorillonite.

The dental composition can be mixed and stored using any one or combination of suitable approaches. For example, a user may place all of the components together, one at a time, or in any combination thereof, on a mixing pad (not shown), in a mixing bowl (not shown), or other mixing apparatus, and then manually mix them using a spatula (not shown) or other mixing tool to initiate polymerization of the dental composition prior to application to the tooth 10. Alternatively, a multi-chamber package, such as a syringe, can house most, if not all, of the components of the dental composition. For example, all the components can be stored in one or the other of two chambers until use of the dental composition is desired.

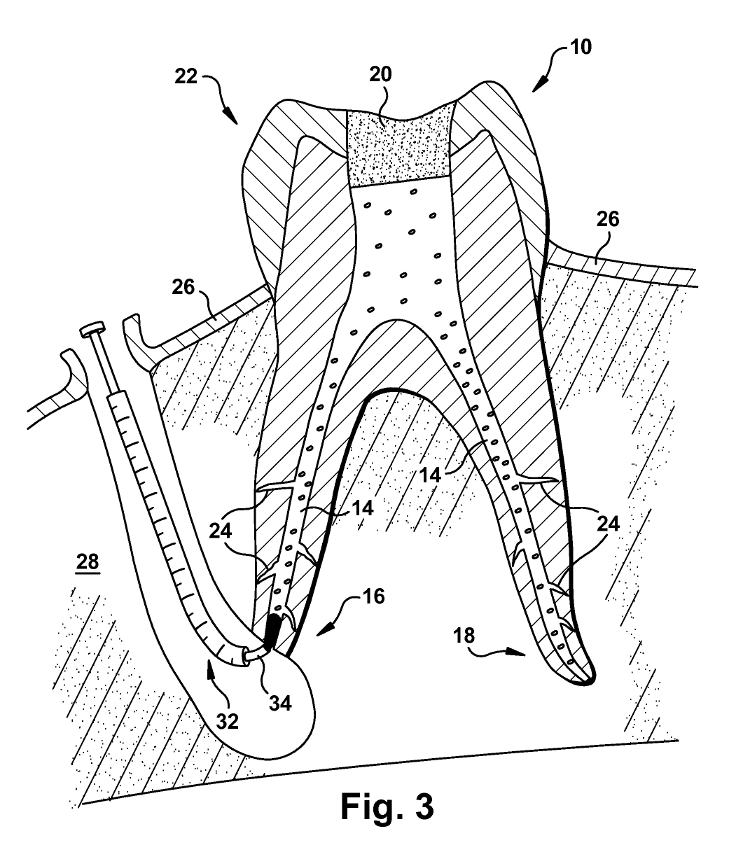

In an example of the method, the components of the dental composition can be manually mixed and then loaded into a syringe 32 (FIG. 3). As shown in FIG. 3, the syringe 32 can have a narrow diameter cannula tip 34 for filling the dental composition into the exposed portion of the first apex 16. Due to the narrow opening of the cannula tip 34, and also because the dental composition may be too viscous to readily pass through the tip, it may be advantageous for the syringe tip to be attached to a high pressure hydraulic injection system (not shown). Examples of high pressure hydraulic syringes or systems are known in the art.

As shown in FIG. 3, the tip 34 of the syringe 32 can initially be placed within the exposed portion of the first apex 16. Prior to placing the syringe 32, however, about 3 to 4 millimeters of the root canal 14 is cleaned by, for example, removing the root filling material and preparing the tooth 10 to receive the dental composition. Next, the dental composition can be dispensed from the syringe 32 into the root canals 14 as shown in FIG. 3. As the dental composition begins and continues to fill up a portion of the root canal 14, the syringe 32 can be slowly raised or withdrawn from the first apex 16. This manner of filling the root canal 14 with the dental composition minimizes or eliminates the formation of air pockets or bubbles as the composition is progressively placed within the root canal. To ensure that the dental composition has penetrated into and sealed every space, irregularity, or lateral canal 24, it may be advantageous to apply additional pressure to the composition after it has been initially placed within the root canal. Additional pressure may be applied by inserting a plunger-like device (not shown), for example, into the just-filled portion of the root canal 14.

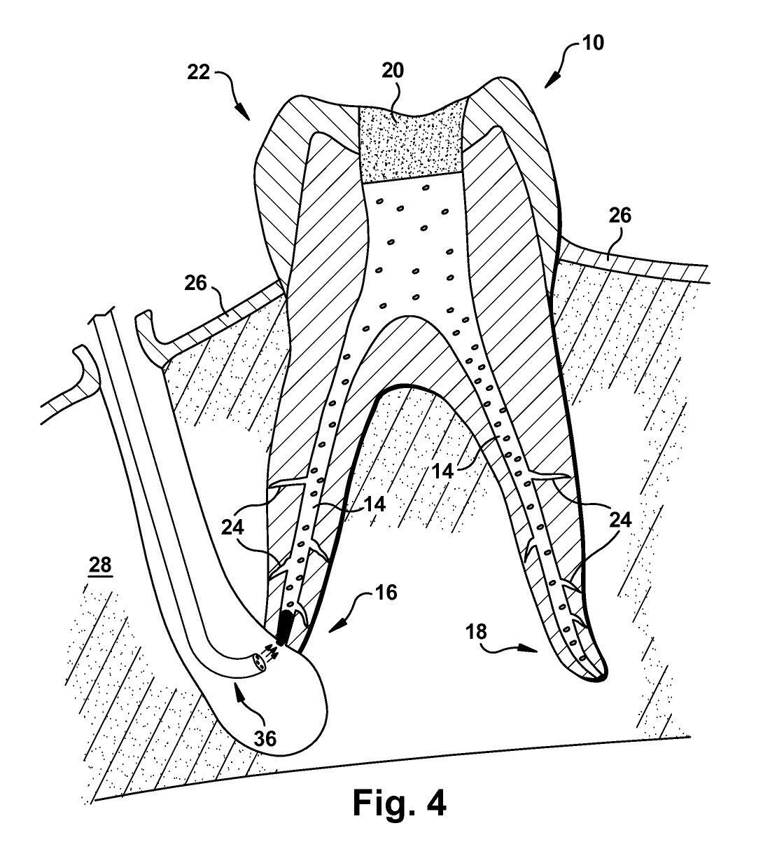

Once the dental composition has been adequately placed in the root canal 14, a radiant energy source 36, such as an ultraviolet curing lamp, can be applied to dental composition as shown in FIG. 4. It will be appreciated that visible light may also be used to cure the dental composition. Applying UV radiation to the dental composition activates the photo-initiator and promotes cross-linking of the liquid acrylic or acrylate monomers with the soluble acrylic or acrylate polymer such that a three-dimensional polymer matrix can be formed.

The matrix formed as a result of photo-curing the dental composition can include an inter-penetrating network (IPN). As used herein, the term “inter-penetrating network or IPN” can include any material containing at least two polymers, each in network form. Several kinds of IPN architectures exist. These systems differ mainly because of the number and types of cross-links that exist in the system. For example, a non-covalent semi-IPN is one in which only one of the polymer systems is cross-linked. Alternatively, a non-covalent full IPN is one in which the two separate polymers are independently cross-linked. A covalent semi-IPN contains two separate polymer systems that are cross-linked to form a single polymer network.

In an example of the present invention, the dental composition can be photo-cured so that the liquid acrylic or acrylate monomer is photo-polymerized in the presence of the acrylic or acrylate polymer, thereby forming a semi-IPN upon photo-polymerization. Photo-curing the dental composition allows for rapid completion of the endodontic procedure while also eliminating the need for a subject to remain in the dental office, for example, while the dental composition is curing. After curing the dental composition, the gum tissue 26 can be repositioned and a few dissolvable sutures (not shown) can be placed to hold the gum tissue back in its place until healing occurs.

The dental composition of the present invention provides a more complete seal of the root canal 14 by increasing the ability of the sealed root canal to resist the ingress of fluids therein and the outflow of the composition into the microporous structure of the tooth 10. Such fluids may enter the root canal 14, for example, through fissures (not shown) in the crown 22 and/or periodontal tissue. Such fluids, if allowed to enter the root canal 14, may carry microbes capable of adhering and colonizing the dental tissue surrounding the root canal. This, in turn, can compromise the mechanical integrity of the tooth 10 and, more importantly, potentially infect the surrounding teeth. In improving the ability of the dental composition to more effectively seal the root canal 14, including the lateral canals 20 and other crevices that are typically hard to fill, the present invention provides a tremendous advantage over conventional compositions and methods in yielding a treated tooth that is more resistant to microbial attack.

The following examples are for the purpose of illustration only and are not intended to limit the scope of the claims, which are appended hereto.

### Example 1

### Synthesis and Characterization of Reactive Clay and Carbon Nanotubes (CNTs)

Ion exchanged organoclays are synthesized by reacting surfactants with hydrophilic clays. A typical procedure is disclosed in Shaikh et al., Annals of Biomedical Engineering, Sep. 1, 2007; PubMed ID: 17786555. Organoclays (e.g., C10, C20A and C30) are purchasable commercially from Southern Clay Products (Gonzales, Tex.).

One possible variation of ion-exchanged clays that are suitable for exfoliation in polymer matrices such as polystyrene, poly(methyl methacrylate), and in rubbers such as in styrene butadiene rubber are reactive clays (see, e.g., Fu, X. et al., Polymer 42(2):807 (2000); Meneghetti, P. et al., Langmuir 20:3424 (2004)). The unique feature of these clays as opposed to commercially available organophilic clays is that they are based on a polymerizable surfactant system. Hence, these are able to better interact and possibly chemical bond to the polymer/rubber matrices. The dispersion of organoclays in the polymer matrix is characterized by X-ray diffraction and transmission electron microscopy (TEM).

Carbon nanotubes are of two varieties based on their structure, single wall nanotubes (SWNT) and multiwall nanotubes (MWNT). One commercial source of SWNTs is Carbon Nanotechnologies Incorporated (Houston, Tex.). Carbon nanotubes synthesized by them using their propriety HIPCO process include “as-synthesized,” purified, and super-purifed grades.

Another synthesis route has been developed that is capable of precise control over the size and surface properties of nanoscale materials (Sankaran, R. M. et al., Nano Lett 537 (5), 2005). The system is a lower power, continuous flow, generic route for nanoparticle growth and manipulation comprised of a plasma microreactor and size classifier.

One of the methods we use is a gas-phase technique utilizing atmospheric pressure microdischarges as short residence time microreactors (Sankaran, R. M. et al., U.S. Pat. No. 6,700,329; Kim, S. H. and Zachariah, M. R., Nanotechnology 16:2149 (2005)). Microdischarges are miniaturized direct-current (dc) plasmas characterized by intense ionization and large concentrations of high-energy electrons. Chemically reactive gases are dissociated in the microplasma through electron impact to produce radical species. At high pressures, the radical species polymerize to nucleate particles homogenously in the gas phase. Particle growth is limited by the small reactor volume (less than 1 nL) crated by the microplasma geometry. Using silane as the precursor, previous experiments have demonstrated the ability to produce optically active Si-nps between the sizes of 1 and 3 nm in a single process.

Application of this technique to the synthesis of metal nanoparticles such as iron allows seeded growth of nanowires and nanotubes. Metal particles are initially grown in the plasma microreactor and introduced into a tube furnace. The addition of acetylene and hydrogen catalyzes the growth of CNTs in free flow (Kim, S. H. and Zachariah, M. R., Nanotechnology 16:2149 (2005)). The gas-phase production of CNTs has two major advantages as compared to traditional growth techniques which grow tubes from a metal film or particles on a substrate. First, aerosol classification can be employed to select a particular diameter and length of nanotube. Second, nanotubes can be modified in-flight to functionalize their surface properties. Since the properties of CNTs depend on both their physical dimensions and their surface chemistry, the synthesis method described here is uniquely capable of tailoring the bulk material properties (e.g., tensile strength) of CNT-based composites.

Methods used for dispersion of the CNTs in the matrix include sonication, homogenization of the CNTs in the monomer, or other physical mixing procedures. For better dispersion in the polymer matrix, the CNTs may be functionalized with functional groups which enhance dispersion in the matrix. The dispersion of the CNTs in the matrix may be characterized by various microscopy procedures such as optical microscopy, SEM or TEM.

### Example 2

### Procedure for Preparing Nanocomposite

The organoparticles were dispersed in a mixture of monomers M3, M4 and M5 along with photo-initiator Init2 and the activator Init3 (Table 1). Polymer P was then added to the monomer-organoclay mix. The weight ratios used for the mixture are displayed in Table 2b. In the control samples (Table 2a), no organoclays were added.

TABLE 1

| | | |
|---|---|---|
| M3 | bis-GMA | 2,2-bis[p-(2-hydroxy-3- |
| | | methacryloxypropoxy)phenyl]propane |
| M4 | TEGDMA | triethylene glycol dimethacrylate |
| M5 | HEMA | hydroxyl ethyl methacrylate |
| M6 | UDMA | urethane dimethacrylate |
| P | PMMA | poly(methyl methacrylate) |
| NC | Organoclay | |
| I2 | CQ | camphorquinone |
| I3 | EDAB | Ethyl-4-N,N-dimethylaminobenzoate |
| I4 | DMAM | N,N-Dimethylaminoethyl Methacrylate |
| I5 | DPC | Diphenyliodonium chloride |
| BPO | | Benzoyl Peroxide |

TABLE 2a

| | % | gms |
|---|---|---|
| Total | 100 | 1 |
| M3 | 45 | 0.45 |
| M4 | 45 | 0.45 |
| M5 | 10 | 0.1 |
| I2 | 0.2 | 0.002 |
| I3 | 0.8 | 0.008 |
| P | 150 | 1.5 |
| NC (% of | 0 | 0 |
| monomer wt) | | |

TABLE 2b

| | % | gms |
|---|---|---|
| Total | 100 | 1 |
| M3 | 45 | 0.45 |
| M4 | 45 | 0.45 |
| M5 | 10 | 0.1 |
| I2 | 0.2 | 0.002 |
| I3 | 0.8 | 0.008 |
| P | 150 | 1.5 |
| NC (% of | 1 | 0.01 |
| monomer wt) | | |

### Polymerization and Pellet Formation Procedure

For the in vitro experiment, the P-M3M4M5-NC mix was placed into the prepared root cavity through a 25-gauge needle/syringe and UV polymerized using a handheld UV curing light for 30 seconds.

TABLE 3

| | % | gms |
|---|---|---|
| Total | 100 | 1 |
| M3 | 45 | 0.45 |
| M4 | 45 | 0.45 |
| M5 | 10 | 0.1 |
| I2 | 0.2 | 0.002 |
| I3 | 0.8 | 0.008 |
| CHX | 2.5 | 0.025 |
| P | 150 | 1.5 |
| NC (% of | 1 | 0.01 |
| monomer wt) | | |

The in vitro drug release study was conducted using pellets of the above mentioned mix (with and without NC) with 2.5 wt % chlorohexidine (CHX) (Table 3). The mix was expressed through the 25-gauge needle into a preformed mould and UV cured for 30 seconds. The pellets were then immersed in 3 mL of sterile water and magnetically stirred for 2 days to evaluate drug release of CHX.

### Procedure for In Vitro Periapical Leakage Assessment

The experimental apparatus was assembled under sterile conditions using a setup modified from Torabinejad et al. (Torabinejad, M. et al., J Endod 21(3):109-112 (1995)). Four milliliter Wheaton glass sample vials (Wheaton-33) were used to fabricate the modified dual chamber apparatus. The vial caps were punctured with a #6 round bur. Each tooth was placed into the fabricated hole in the cap so that the coronal end of the canal space was continuous with the hole. The teeth were secured to vial caps with cyanoacrylate. Following the completion of assembly, teeth were sterilized with gamma radiation (Cesium-137).

The roots were suspended in sterile BHI broth and the entire apparatus was placed into the incubator at 37° C. for 24 hours. After 24 hours, all samples were observed and any turbid samples were considered contaminated and discarded.

A pure isolate of the Gram-positive facultative anaerobe E. faecalis (ATCC 29212j) was inoculated into a 10 mL vial of sterile BHI broth and placed into the incubator for 24 hours at 37° C. A sample of the bacteria culture was tested using Gram staining to verify the purity of the inoculum after the culture medium became turbid. Under the light microscope, the E. faecalis sample stained Gram-positive (purple) diplococci. From this bacterial sample, 100 μL was taken and inoculated into a new sterile tube of BHI broth. This was continued each day so that fresh E. faecalis was used daily to inoculate the experimental groups.

All samples were inoculated with 50 μL of E. faecalis using a micropipette. Each consecutive day, a fresh E. faecalis (50 μL) was placed into the coronal surface of the root. All samples were examined for turbidity. When a sample became turbid, Gram-staining was completed from the turbid broth to confirm the presence of E. faecalis. Day of turbidity was recorded for each sample for 30 days.

### Example 3

### Leakage Assessment

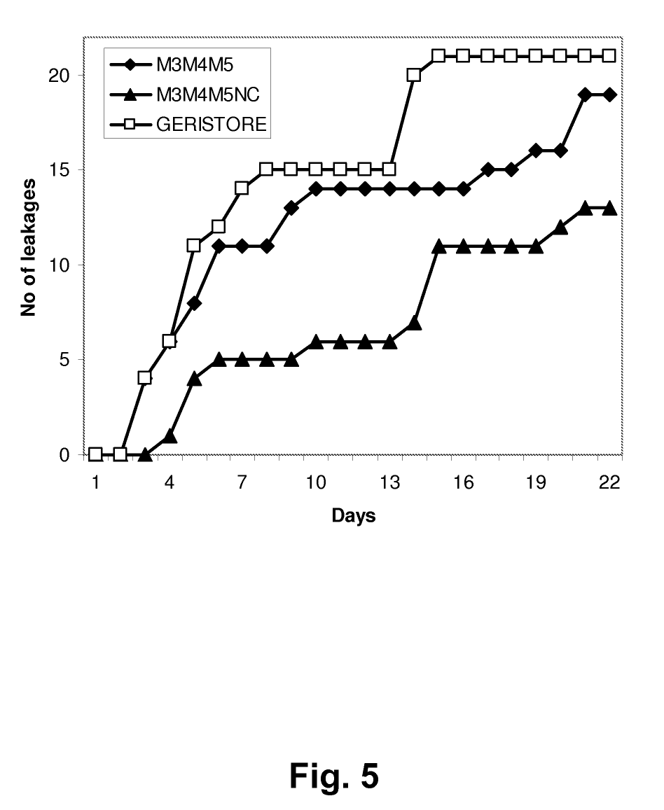

A leakage study was performed comparing sealability of polymer nanocomposite, control, and commercially available compomer (GERISTORE) used currently as root end filling material. The results are shown in Table 4 and FIG. 5.

TABLE 4

| DAY | M3M4M5 | M3M4M5NC | GERISTORE |
|---|---|---|---|
| 0 | 0 | 0 | 0 |
| 1 | 4 | 0 | 4 |
| 2 | 6 | 1 | 6 |
| 3 | 8 | 4 | 11 |
| 4 | 11 | 5 | 12 |
| 5 | 11 | 5 | 14 |
| 6 | 11 | 5 | 15 |
| 7 | 13 | 5 | 15 |
| 8 | 14 | 6 | 15 |
| 9 | 14 | 6 | 15 |
| 10 | 14 | 6 | 15 |
| 11 | 14 | 6 | 15 |
| 12 | 14 | 7 | 20 |
| 13 | 14 | 11 | 21 |
| 14 | 14 | 11 | 21 |
| 15 | 15 | 11 | 21 |
| 16 | 15 | 11 | 21 |
| 17 | 16 | 11 | 21 |
| 18 | 16 | 12 | 21 |
| 19 | 19 | 13 | 21 |
| 20 | 19 | 13 | 21 |

The test data indicated that the nanocomposite system may be used as a controlled release drug delivery system whereby drug release from the composition is slowed, expedited, or otherwise manipulated in comparison to the non-clay containing system.

### Example 4

### Drug Efficacy

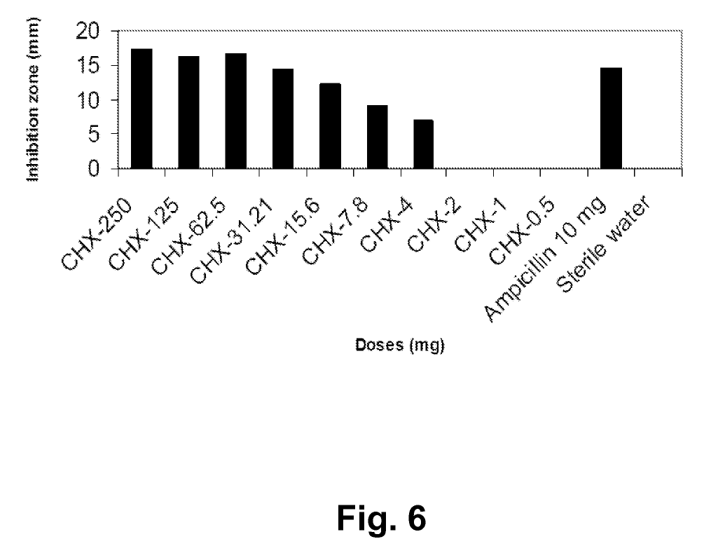

The efficacy of the eluted amounts of CHX was tested to evaluate anti-microbial activity at various dose strengths compared to a positive control (Ampicillin). This pilot study was done to obtain a range of CHX elution amounts by weight required to kill common bacteria associated with failed root canal therapy. Table 5 demonstrates that even at 4 μg of CHX, some antimicrobial effect can be seen (which is more than water, the negative control). Interestingly, FIG. 6 shows that the zones of inhibition for 31 to 250 μg of CHX are equal to or better than that of Ampicillin.

TABLE 5

| Group/Dose (μg) | Zones (mm) |
|---|---|
| CHX-250 | 17.3 |
| CHX-125 | 16.3 |
| CHX-62.5 | 16.7 |
| CHX-31.25 | 14.3 |
| CHX-15.6 | 12.3 |
| CHX-7.8 | 9 |
| CHX-4 | 7 |
| CHX-2 | 0 |
| CHX-1 | 0 |
| CHX-0.5 | 0 |
| Ampicillin 10 mg | 14.5 |
| Sterile water | 0 |

### Example 5

### Qualitative Assessment of a Nanocomposite-Dentin Interface Using Scanning Electron Microscopy

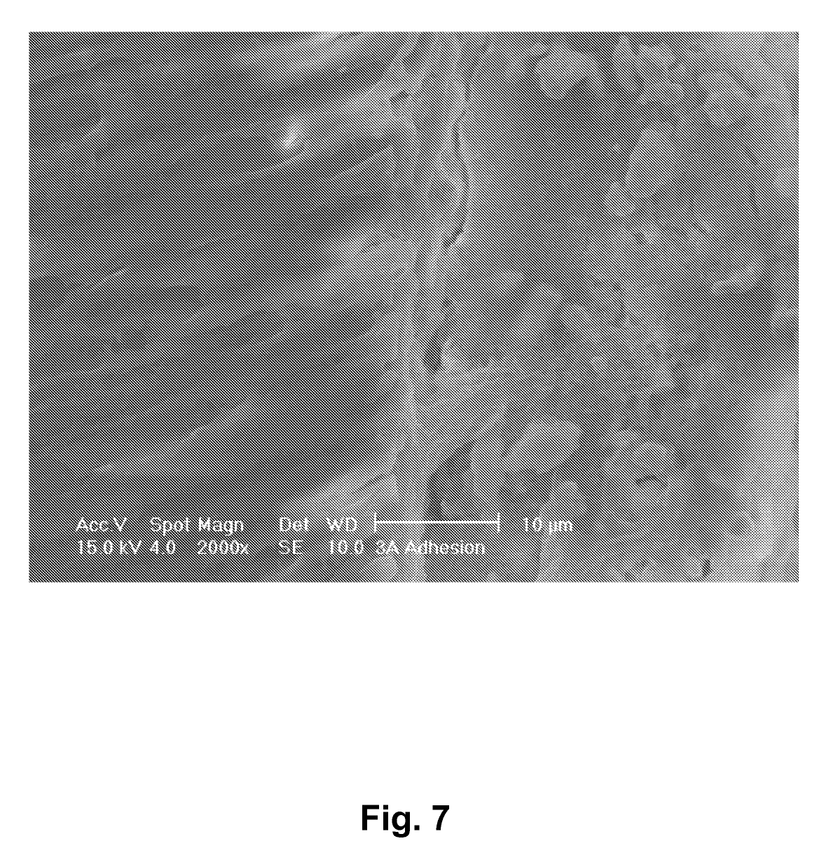

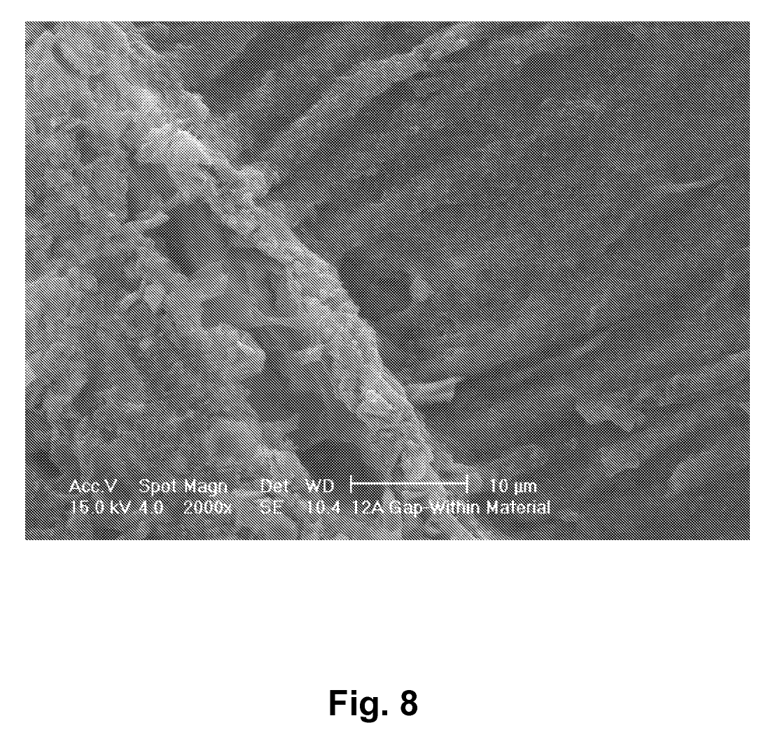

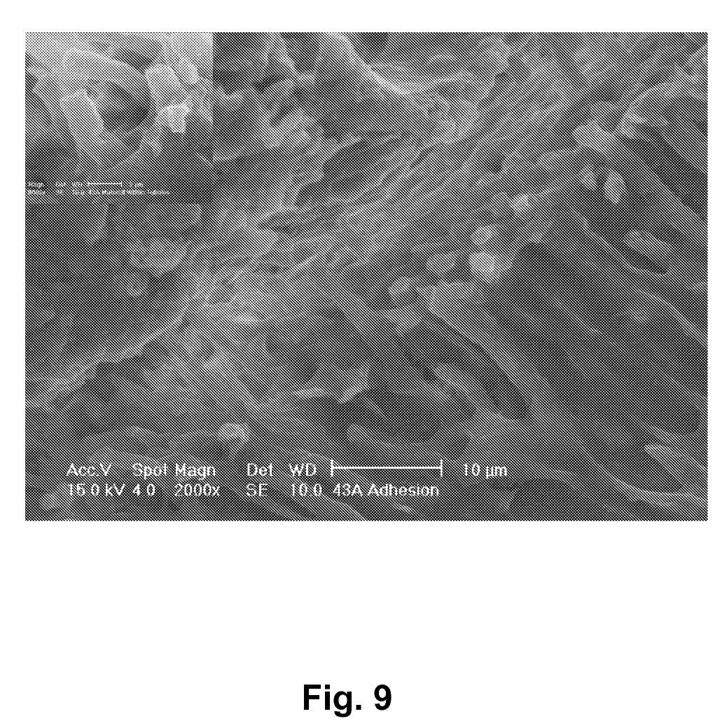

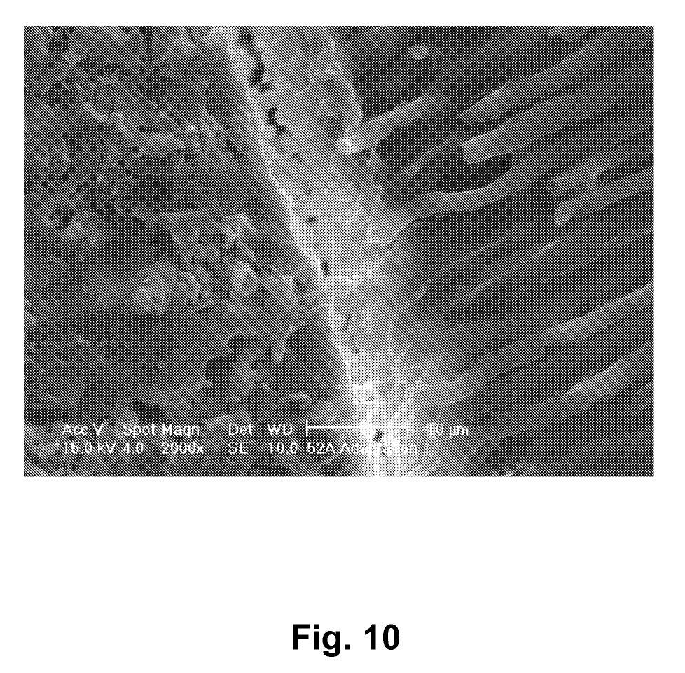

The purpose of this study was to qualitatively assess the dentin-material interface of root canals filled with PNCs using SEM as compared to Mineral Trioxide Aggregate (MTA) and Geristore™. Thirty-two single-rooted extracted teeth were decoronated and biomechanically prepared. The apical 3 mm of each tooth was resected and the remaining canal space was filled with MM, MM+1% nanoparticle (C18), MM+2% C18, MM+1% C18+1% chlorhexidine (CHX), MM+2% C18+2% CHX, MTA, or GERISTORE. The MM consisted of Bis-GMA+TEGDMA+HEMA monomers. The filled canals were sectioned into three 2.0 mm sections using the Isomet 1000 Precision SAW. Apical sections were then prepared and analyzed using the Philips XL30 SEM (middle and coronal sections were used for parallel studies). SEM micrographs of the respective samples are in FIGS. 7-10. FIGS. 7-10 show that although tubule occlusion at the interface was found using MTA and GERISTORE, tubule penetration (>50%) was more prevalent in the samples filled with MM alone and MM+1% or 2% C18. Smear layer removal increased penetration as well, but adding CHX to the mixture reduced tubule penetration (<50%) compared to groups without CHX.

### Example 6

### Evaluation of Bond Strength of New Nano-Clay Enhanced Retrofill Polymer Material in Endodontics

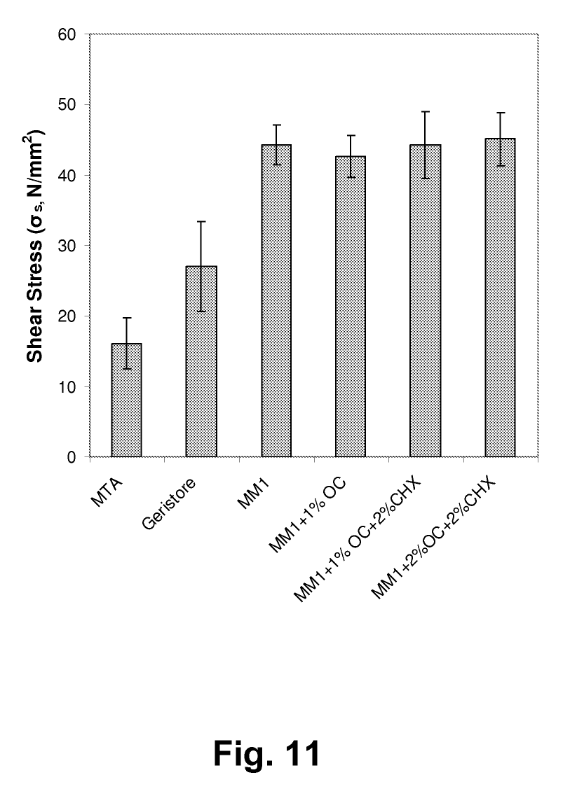

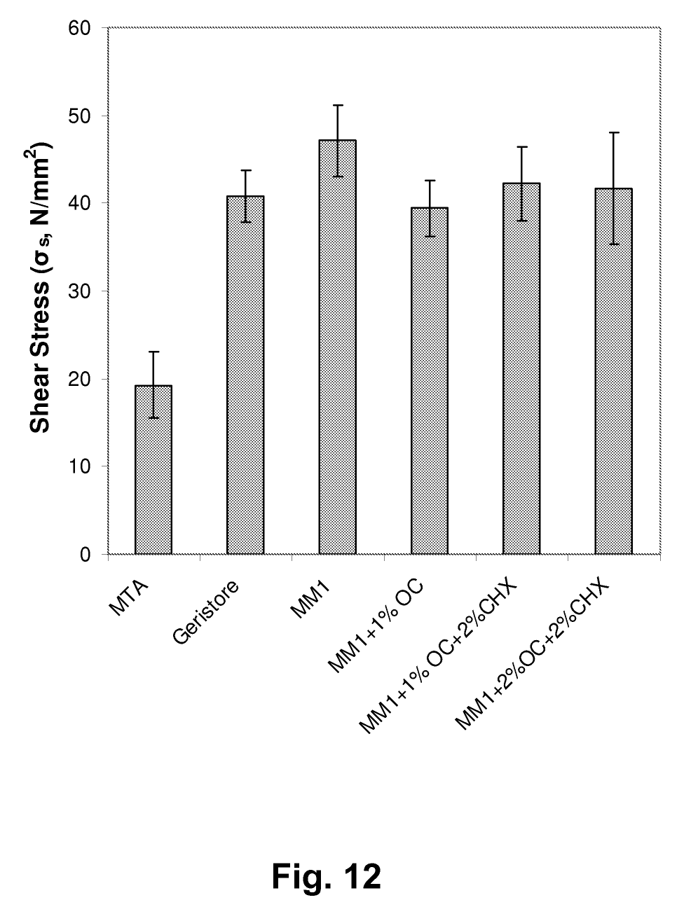

The objective of this study was to evaluate push-out bond strength (PBS) of polymer nanocomposites (PNCs) to dentin and characterize the mode of failure at the PNC/dentin-interface. Analyzed groups of PNCs included: PM+1% OC, PM+1% OC+2% Chlorohexidine (CHX), PM+2% OC+2% CHX. These were compared to MTA and GERISTORE. Eighty extracted single-rooted teeth were decoronated, endodontically prepared, apically resected, and sub-grouped based on smear layer removal. Roots were filled with one of the materials and sectioned into 2 mm sections with 26 samples per group. PBS testing was performed using a universal testing device (Instron) and failure method was analyzed using light microscopy and a scanning electron microscope (SEM). Microscopic examination of the samples showed adhesive failure occurred in nearly every sample. FIG. 11 illustrates a chart showing shear stress of PNC samples compared to MTA and GERISTORE in the presence of a dentinal smear lay. FIG. 12 illustrates a chart showing shear stress for PNC samples compared to MTA and GERISTORE in the absence of dentinal smear layer. Analysis of variance (ANOVA) test results (alpha=0.05) on push-out samples showed significant differences in recorded failure stress values (p<0.0001) between materials. Pair wise t-testing showed PNC groups required significantly larger (p<0.03) stress values to push out the material than MTA and Geristore when smear layer was intact. In the absence of dentinal smear layer, the PNC groups showed significantly higher stress values compared to MTA but not GERISTORE. In the current study, the bond strength of PNC groups was shown to be higher than MTA and GERISTORE.

### Example 7

### XRD Analysis of Nanoparticle Dispersion in a Novel Endodontic Polymer

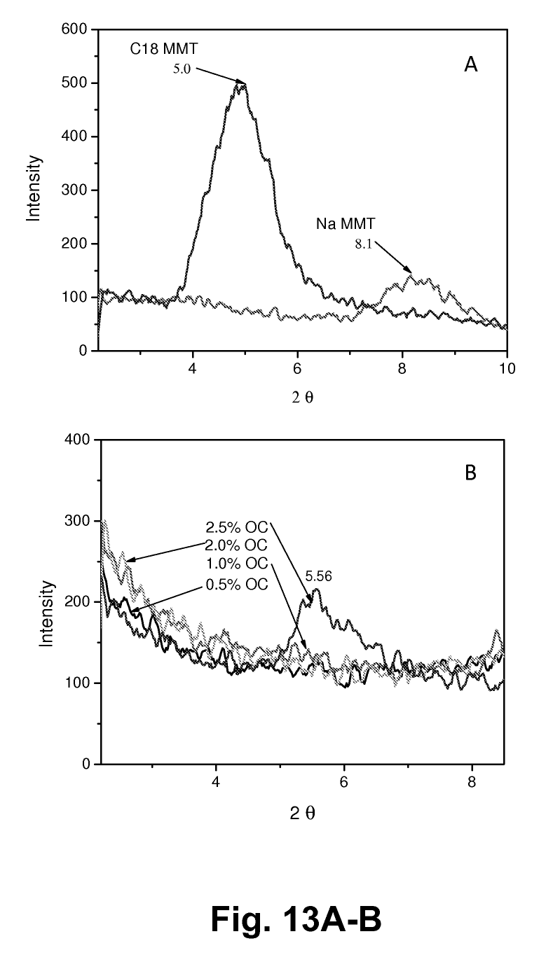

We quantitatively characterized the exfoliation of two different types of organoclays within a specific polymer-monomer matrix using X-ray Diffraction. Two different species of organoclays were incorporated into a dental monomer matrix in concentrations of 0.5%, 1%, 1.5%, 2%, and 2.5% by weight. The matrix was composed of bisphenol A glycerolate(1 glycerol/phenol)dimethacrylate (Bis-GMA), triethylene-glycol-dimethacrylate (TEGDMA), and 2-hydroxyethyl methacrylate (HEMA). A film with thickness of 0.5 mm was prepared of the sample for x-ray diffraction, with data being analyzed for patterns associated with exfoliation and compared to known patterns found in Na-MMT. As evidenced by the XRD results in FIGS. 13A and 13B, exfoliation was obtained in all samples with an organoclay concentration of 2% or less. This study demonstrates that organoclay based polymer nanocomposites are a viable option for future development and use as an endodontic retrofill material.

### Example 8

### TEM Analysis of Nanoparticle Dispersion in a Novel Endodontic Polymer

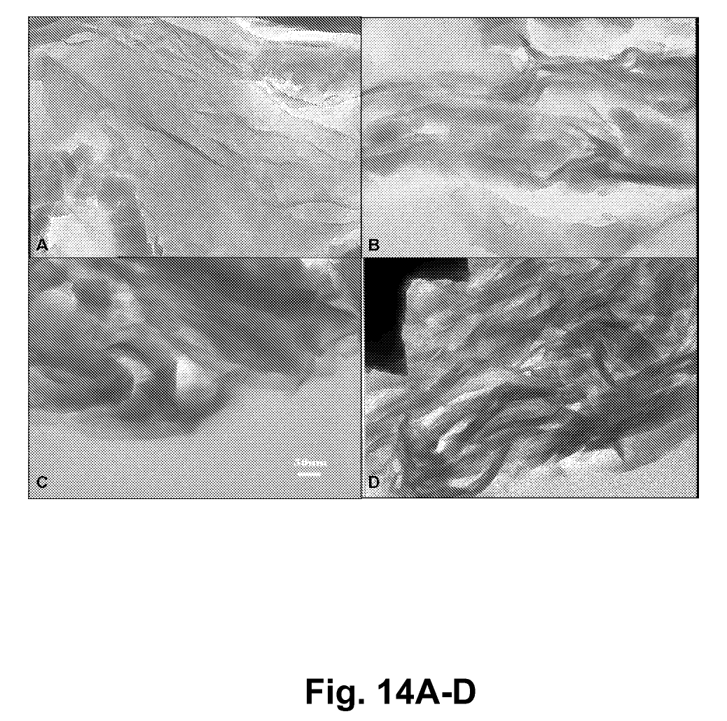

This study investigates the use of PNCs as potentially useful materials in Endodontic surgery and to determine qualitatively the degree of dispersion of various nanoparticles namely, organoclays (OC), carbon nanotubes (CNT), and Graphene in a dental monomer matrix using Transmission Electron Microscopy. Nanoparticles were mixed with Bis-GMA/TEGDMA/HEMA monomer resins to study the degree of dispersion (intercalation or exfoliation). Samples were photo-polymerized and sectioned by ultra-microtome. Nanoparticle dispersion in samples was examined by TEM. FIG. 14 illustrates TEM Micrographs of PNCs at varying OC content (50 nm scale) (A) 0.5% OC (B) 1.0% OC(C) 1.5% OC (D) 2.0% OC. TEM micrographs reveal that smaller mass fractions of OC (0.5% and 1.0%) in the Bis-GMA/TEGDMA/HEMA system (40/40/20 mass ratio) demonstrated a high degree of exfoliation. When larger mass fractions of OC were used, these resulted in partially intercalated/exfoliated nanocomposites. Studies with CNT and Graphene also showed a homogenous dispersion of nanoparticles. From the TEM studies, it can be observed that OC particles at lower loading appear to be highly exfoliated in the polymer matrix. Good dispersion has also been achieved using carbon nanotubes and graphene.

### Example 9

### NMR Analysis of Dental Nanocomposite Precursors

Proton nuclear magnetic resonance (NMR) spectroscopy was used to quantify monomers released from dental composite precursors. Composite precursors were made using three monomers, four initiators and clay as nano-filler. Monomers used were 2-Hydroxyethyl methacrylate (HEMA), Triethylene glycol dimethacrylate (TEGDMA) and Bisphenol A glycerolate dimethacrylate (BisGMA). Initiators used were Camphorquinone (CQ), Ethyl 4-(dimethylamino)benzoate (EDAB), 2-(Dimethylamino)ethyl methacrylate (DMAEMA) and Diphenyliodonium chloride (DC). Clay used was Na-montmorillonite (Na-MMT) and organically modified form of the same clay (C18-MMT). Five batches were made; clay-free batch (control), batches with 1% and 2% Na-MMT and batches with 1% and 2% C18-MMT. Quantitative NMR experiments were carried using trimethylbenzene (TMB) as internal standard. All five batches released similar amounts of residual monomer (4.47e−02 moles/g±1.76e−02 (SD)) indicating that polymerization kinetics is not affected by clay type or concentration.

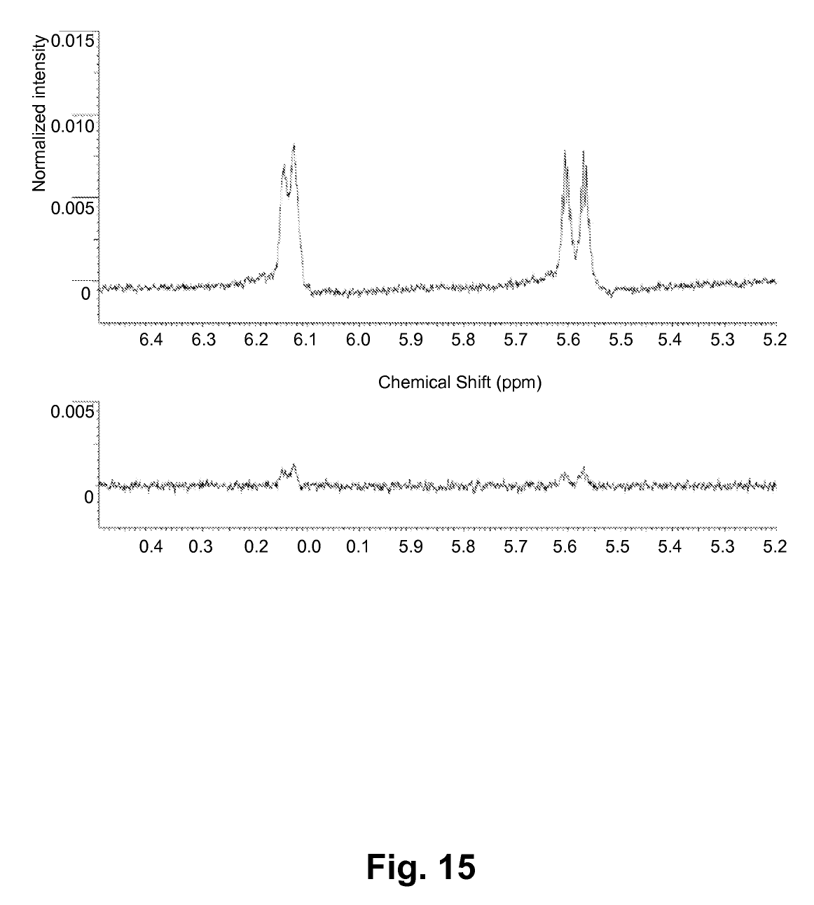

FIG. 15 illustrates ¹H NMR spectra comparison of MM1+I2I3 (top spectrum) and MM 1+I2I3I4I5 (bottom spectrum. It was shown that set I2I3I4I5 of initiators showed a pronounce decrease in monomers release in a given system. The spectra show the protons of carbon-carbon double bond, which is present in HEMA, BisGMa and TEGDMA. When polymerization occur, these double bonds become single bond and this can be detected using NMR. The presence of these double bonds is an indication of the presence of these monomers and the area and magnitude of proton peaks are quantitative assessment of molecule concentration. It was concluded from this qualitative comparison that set I2I3I4I5 is much better group of initiators that will minimize the residual monomer in the dental composite.

### Example 10

### Organic Leachables in Methacrylate Based Resin Composite Samples by UV-Vis Spectroscopy

Samples of light cured polymer of proposed mass (200 mg per application) to be used in endodontic procedures were extracted by shaking overnight with 50% ethanol, phosphate buffer pH 7.4, acetate buffer pH 4.6 or 0.9% saline. The extract mixtures were measured by UV-visible spectra from 200 to 400 nm to provide a rapid and simple method to quantitatively measure selected unbound components. Concentrations were estimated by comparison to spectra of serially diluted samples of individual components in selected solvents.

Acute toxicity levels were obtained from material safety data sheets. The LD50 values for measured components include cross linking monomers Bisphenol A-glycidyl methacrylate (M3) LD50>5000 mg/Kg (RAT), Triethylene Glycol Dimethacrylate (M4) 10750 mg/kg, 2-Hydroxyethyl methacrylate (M5) 4680 mg/kg, initiators Ethyl 4-(dimethylamino)benzoate (I3) 2000 mg/Kg (RAT), 2-(Dimethyleamino)Ethyl Methacrylate (I4) 1751 mg/Kg RAT ORAL, and Diphyenyliodonium Chloride (I5) 60 mg/kg and antimicrobial agent chlorhexidine (CHX) 2000 mg/Kg. Amounts of M3, M4, M5 and CHX potentially leaching from a 200 mg sample (10 times clinical dose) were well below (at least 4 orders of magnitude) acutely toxic levels for a 70 Kg patient (Table 6). Addition of organoclay nanoparticles to the formulation of methacrylate based retrofill material did not change the levels of leachable compounds recovered from samples. Addition of M5 to the composite formulation appeared to reduce the amount of M4 extracted from cured samples.

TABLE 6

| | M3 Total | M4 Total | M5 Total | CHX Total |
|---|---|---|---|---|
| | Extracted | Extracted | Extracted | Extracted |
| | Mass (mg) | Mass (mg) | Mass (mg) | Mass (mg) |
| Sample Description | (Ethanol) | (Ethanol) | (Ethanol) | (Ethanol) |
| 0% NC, 0% CHX, M3, M4, M5, 12, 13, 14, 15, BPO | 2.4 | 4.1 | 3.0 | na |
| 1% NC, 0% CHX, M3, M4, M5, 12, 13, 14, 15, BPO | 3.5 | 4.1 | 3.1 | na |
| 1% NC, 2% CHX, M3, M4, M5, 12, 13, 14, 15, BPO | 4.7 | 4.2 | 3.1 | 4.0 |
| 2% NC, 0% CHX, M3, M4, M5, 12, 13, 14, 15, BPO | 4.2 | 4.1 | 3.1 | na |
| 0% NC, 0% CHX, M3, M4, 0% M5, 12, 14, 15, 17, BPO | 4.5 | 4.2 | na | na |
| 0% NC, 0% CHX, M3, M4, 0% M5, 12, 13, 0% 14, 0% 15, BPO | 4.8 | 4.3 | na | na |
| 0% NC, 1% CHX, M3, M4, 0% M5, 12, 13, 0% 14, 0% 15, BPO | 6.5 | 4.4 | na | 1.5 |
| 0% NC, 1% CHX, M3, M4, 0% M5, 12, 13, 14, 15, BPO | 6.3 | 4.4 | na | 1.5 |
| | M3 Total | M4 Total | M5 Total | CHX Total |
| | Extracted | Extracted | Extracted | Extracted |
| | Mass (mg) | Mass (mg) | Mass (mg) | Mass (mg) |
| Sample Description | (acetate) | (acetate) | (acetate) | (acetate) |
| 0% NC, 0% CHX, M3, M4, M5, 12, 13, 14, 15, BPO | 0.0 | 2.6 | 2.3 | none added |
| 1% NC, 0% CHX, M3, M4, M5, 12, 13, 14, 15, BPO | 0.0 | 2.9 | 3.0 | none added |
| 1% NC, 2% CHX, M3, M4, M5, 12, 13, 14, 15, BPO | 0.0 | 2.7 | 2.6 | 0.2 |
| 2% NC, 0% CHX, M3, M4, M5, 12, 13, 14, 15, BPO | 0.0 | 3.0 | 3.4 | 0.1 |
| 0% NC, 0% CHX, M3, M4, 0% M5, 12, 13, 14, 15, BPO | 0.0 | 2.8 | none added | none added |
| 0% NC, 0% CHX, M3, M4, 0% M5, 12, 13, 0% 14, 0% 15, BPO | 0.0 | 2.3 | none added | none added |
| 0% NC, 1% CHX, M3, M4, 0% M5, 12, 13, 0% 14, 0% 15, BPO | 0.0 | 2.7 | none added | 0.1 |
| 0% NC, 1% CHX, M3, M4, 0% M5, 12, 13, 14, 15, BPO | 0.0 | 2.8 | none added | 0.1 |
| | M3 Total | M4 Total | M5 Total | CHX Total |
| | Extracted | Extracted | Extracted | Extracted |
| | Mass (mg) | Mass (mg) | Mass (mg) | Mass (mg) |
| Sample Description | (Phosphate) | (Phosphate) | (Phosphate) | (Phosphate) |
| 0% NC, 0% CHX, M3, M4, M5, 12, 13, 14, 15, BPO | 0.0 | 1.8 | 1.9 | na |
| 1% NC, 0% CHX, M3, M4, M5, 12, 13, 14, 15, BPO | 0.0 | 3.2 | 3.4 | na |
| 1% NC, 2% CHX, M3, M4, M5, 12, 13, 14, 15, BPO | 0.0 | 2.5 | 2.6 | 0.1 |
| 2% NC, 0% CHX, M3, M4, M5, 12, 13, 14, 15, BPO | 0.0 | 3.0 | 3.2 | 0.1 |
| 0% NC, 0% CHX, M3, M4, 0% M5, 12, 13, 14, 15, BPO | 0.0 | 2.6 | na | na |
| 0% NC, 0% CHX, M3, M4, 0% M5, 12, 13, 0% 14, 0% 15, BPO | 0.0 | 2.1 | na | na |
| 0% NC, 1% CHX, M3, M4, 0% M5, 12, 13, 0% 14, 0% 15, BPO | 0.0 | 2.1 | na | 0.1 |
| 0% NC, 1% CHX, M3, M4, 0% M5, 12, 13, 14, 15, BPO | 0.0 | 2.7 | na | Na |
| | M3 Total | M4 Total | M5 Total | CHX Total |
| | Extracted | Extracted | Extracted | Extracted |
| | Mass (mg) | Mass (mg) | Mass (mg) | Mass (mg) |
| Sample Description | (Saline) | (Saline) | (Saline) | (Saline) |
| 0% NC, 0% CHX, M3, M4, M5, 12, 13, 14, 15, BPO | 0.0 | 2.4 | 2.6 | none added |
| 1% NC, 0% CHX, M3, M4, M5, 12, 13, 14, 15, BPO | 0.0 | 3.0 | 3.9 | none added |
| 1% NC, 2% CHX, M3, M4, M5, 12, 13, 14, 15, BPO | 0.0 | 2.5 | 2.7 | 0.03 |
| 2% NC, 0% CHX, M3, M4, M5, 12, 13, 14, 15, BPO | 0.0 | 3.1 | 4.0 | none added |
| 0% NC, 0% CHX, M3, M4, 0% M5, 12, 13, 14, 15, BPO | 0.0 | 2.7 | none added | none added |
| 0% NC, 0% CHX, M3, M4, 0% M5, 12, 13, 0% 14, 0% 15, BPO | 0.0 | 2.8 | none added | none added |
| 0% NC, 1% CHX, M3, M4, 0% M5, 12, 13, 0% 14, 0% 15, BPO | 0.0 | 2.9 | none added | 0.1 |
| 0% NC, 1% CHX, M3, M4, 0% M5, 12, 13, 14, 15, BPO | 0.0 | 3.1 | none added | 0.1 |

From the above description of the invention, those skilled in the art will perceive improvements, changes and modifications. Such improvements, changes and modifications are within the skill of the art and are intended to be covered by the appended claims. All patents publications and references cited in the present application are herein incorporated by reference in their entirety.

Having described the invention, we claim:

1. A dental composition for sealing a portion of a tooth comprising:
a liquid acrylic or acrylate monomer;
an acrylic or acrylate polymer that is at least partially soluble in the liquid acrylic or acrylate monomer;
a photo-initiator for cross-linking the liquid acrylic or acrylate monomer; and
a nanoparticle material dispersed within the composition.

2. The composition of claim 1, the initial and long-term leakage of the dental composition from a portion of the tooth being prevented or mitigated.

3. The composition of claim 1, the liquid acrylic or acrylate monomer selected from the group consisting of methyl methacrylate, butyl methacrylate, 2,2-bis[p-(2-hydroxy-3-methacryloxypropoxy)phenyl]propane (bisGMA), triethylene glycol dimethacrylate (TEGDMA), urethane dimethacrylate (UDMA), and hydroxyl ethyl methacrylate (HEMA).

4. The composition of claim 1, the liquid acrylic or acrylate monomer comprising about 20% to about 60% by weight of the dental composition.

5. The composition of claim 1, the soluble acrylic or acrylate polymer comprising poly(methyl methacrylate).

6. The composition of claim 1, the soluble acrylic or acrylate polymer comprising about 10% to about 200% by weight of the dental composition.

7. The composition of claim 1, the nanoparticle material comprising an organically-modified clay selected from the group consisting of montmorillonite, fluorohectorite, bentonite, fluoromica, and layered double hydroxides.

8. The composition of claim 1, the nanoparticle material comprising a carbon nanotube.

9. The composition of claim 1 further including at least one anti-microbial agent.

10. A dental composition for sealing a portion of a tooth comprising:
a liquid acrylic or acrylate monomer comprising about 20% to about 60% by weight of the dental composition;
an acrylic or acrylate polymer that is soluble in the liquid acrylic or acrylate monomer comprising about 10% to about 200% by weight of the dental composition;
a photo-initiator for cross-linking the liquid acrylic or acrylate monomer; and
a nanoparticle material dispersed within the composition.

11. The composition of claim 10, the initial and long-term leakage of the dental composition from a portion of the tooth being prevented or mitigated.

12. The composition of claim 10, the liquid acrylic or acrylate monomer selected from the group consisting of methyl methacrylate, butyl methacrylate, bisGMA, TEGDMA, UDMA and HEMA.

13. The composition of claim 10, the soluble acrylic or acrylate polymer comprising poly(methyl methacrylate).

14. The composition of claim 10, the nanoparticle material comprising an organically-modified clay selected from the group consisting of montmorillonite, fluorohectorite, bentonite, fluoromica, and layered double hydroxides.

15. The composition of claim 10, the nanoparticle material comprising a carbon nanotube.

16. The composition of claim 10 further including at least one anti-microbial agent.

17. A method for sealing a portion of a tooth, the method comprising the steps of:
administering to the portion of the tooth at least one application of a dental composition comprising a liquid acrylic or acrylate monomer, an acrylic or acrylate polymer that is soluble in the liquid acrylic or acrylate monomer, a photo-initiator, and a nanopolymer material; and photo-curing the dental composition so that the liquid acrylic or acrylate monomer and the soluble acrylic or acrylate polymer are cross-linked and form a three-dimensional polymer matrix having the nanoparticle material dispersed therein.

18. The method of claim 17, the initial and long-term leakage of the dental composition from a portion of a tooth being prevented or mitigated.

19. The method of claim 17, the liquid acrylic or acrylate monomer selected from the group consisting of methyl methacrylate, butyl methacrylate, bisGMA, TEGDMA, UDMA and HEMA.

20. The method of claim 17, the liquid acrylic or acrylate monomer comprising about 20% to about 60% by weight of the dental composition.

21. The method of claim 17, the soluble acrylic or acrylate polymer comprising poly(methyl methacrylate).

22. The method of claim 17, the soluble acrylic or acrylate polymer comprising about 10% to about 200% by weight of the dental composition.

23. The method of claim 17, the nanoparticle material comprising an organically-modified clay selected from the group consisting of montmorillonite, fluorohectorite, bentonite, fluoromica, and layered double hydroxides.

24. The method of claim 17, nanoparticle material comprising a carbon nanotube.

25. The method of claim 17, the dental composition further including at least one anti-microbial agent.
